(19) United States
(12) Patent Application Publication
(10) Pub. No.: US 2002/0045207 A1
(43) Pub. Date: Apr. 18, 2002

(54) GLYCOPROTEIN PRODUCTION PROCESS

(76) Inventors: LYNNE A. KRUMMEN, SAN FRANCISCO, CA (US); MARY B. SLIWKOWSKI, SAN CARLOS, CA (US); TOM WARNER, SAN CARLOS, CA (US)

Correspondence Address: KATHERINE M. KOWALCHYK, MERCHANT AND GOULD, P.O.BOX 2903, MINNEAPOLIS, MN, 55402-0903, US

(21) Appl. No.: 09/175,202

(22) Filed: Oct. 19, 1998

Related U.S. Application Data
(60) Provisional application No. 60/063,872, filed on Oct. 31, 1997.

(51) Int. Cl. C12P 21/06

(52) U.S. Cl. 435/69.1; 435/193

(57) ABSTRACT

Glycoprotein production methods and host cell lines are provided. The production methods and host cells include the functional expression of a galactosyltransferase and a sialyltransferase alone or in combination with a host cell line selected for lack of functional expression of a glycohydrolytic enzyme such as a sialidase.

### BACKGROUND OF THE INVENTION

1. Field of the Invention

This invention relates generally to the field of glycoprotein production by cell culture. In particular aspects the invention relates to glycoprotein production methods and recombinant host cells for the production of human therapeutics.

2. Description of Related Disclosures

Differences in glycosylation patterns of recombinantly produced glycoproteins have recently been the focus of attention in the scientific community as recombinant proteins produced as probable prophylactics and therapeutics approach the clinic. Oligosaccharide structures on glycoproteins produced by expression of foreign genes in recombinant host cells in vitro are heterogenous in both their composition and structure (Rademacher et al. (1988) Annu. Rev. Biochem. 57:785-838; Spellman et al., (1989) J. Biol. Chem. 264(24):14100-14111; Spellman et al., (1991) Biochemistry 30:2395-2406; Kagawa et al., (1988) J. Biol. Chem. 263(33):17508-17515).

The oligosaccharide side chains of the glycoproteins affect the protein's function (Wittwer A., and Howard, S. C. (1990) Biochem. 29:4175-4180) and the intramolecular interaction between portions of the glycoprotein resulting in the conformation and presented three dimensional surface of the glycoprotein (Hart, (1992) Curr. Op. Cell Biol., 4:1017-1023; Goochee, et al., (1991) Bio/Technology, 9:1347-1355; Parekh, R. B., (1991) Curr. Op. Struct. Biol., 1:750-754). In particular, the terminal sialic acid component of the glycoprotein oligosaccharide side chain affects absorption, serum half life, and clearance from the serum, as well as the physical, chemical and immunogenic properties of the glycoprotein (Parekh, R. B., supra; Varki, A., (1993) Glycobiology 3:97-100; Paulson, J. (1989), TIBS, 14:272276; Goochee, et al., (1991) Biotechnology 9:1347-1355; Kobata, A, (1992) Eur. J. Biochem. 209:483-501). As a result the molar content of sialic acid on a recombinant glycoprotein can be a key feature of the therapeutic glycoprotein's biological quality.

Several strategies have been proposed to affect the composition of oligosaccharides in recombinant glycoproteins (Minch SL, et al. (1995) Biotechnol Prog 11:348-351; Monaco L, et al. (1996) Cytotechnology 22:197-203; Grabenhorst et al., (1995) Eur. J. Biochem. 232:718-725; U.S. Pat. No. 5,047,335). Expression of human α2,6-sialyltransferase in recombinant cell lines such as Chinese hamster ovary (CHO) cells and baby hamster kidney cells (BHK) has been shown to alter the quality of the N-linked oligosaccharide terminal glycoside linkage producing a more “human” product ( Minch et al., supra; Monaco et al., supra;). The transfected sialyltransferase can compete with endogenous CHO cell enzymes for glycosyl substrate in the attachment of terminal sugar residues (Lee UE, et al. (1989) J Biol Chem 264:13848-13855). Expression of other glycosyltransferases in recombinant host cell lines has also been reported (Miyoshi E, et al. (1995) J. Biol. Chem. 270(47):28311-28315; Ernst et al., (1989) J. Biol. Chem. 264(6):3436-3447). These studies support the potential of genetic strategies directed toward oligosaccharide biosynthesis for altering the glycoprotein production.

Other strategies include the prevention of oligosaccharide degradation by glycohydrolytic enzymes such as sialidases which can cleave sialic acid residues from the oligosaccharide components of glycoproteins and glycolipids (Sliwkowski et al., (1992) J. Cell. Biochem. 16D:159; Gramer M J, et al. (1995) Biotechnology 13:692-697). Various sialidases have been identified (Air, G. M. and Laver, W. G. (1989) Proteins: Struct. Func. Genet., 6:341; Warner T G, et al. (1993) Glycobiology 3(5):455-463; Ferrari J, et al. (1994) Glycobiology 4(3): 367-373). Sialidases have been identified in a number of cellular organelles including the plasma membrane (Schengrund, C. et al. (1976) Journal of Cell Biology, 70:555), the lysosomes and the cytosol (Tulsiani, D. R. P., and Carubelli, R., (1970) J. Biol. Chem., 245:1821).

For glycoproteins whose half-life or biological activity is strongly dependent on the content of sialic acid, insufficient or inconsistent glycosylation is a significant problem for adequate, reproducible dosing of the molecule. From a manufacturing perspective, since the degree of glycosylation can vary as a function of environmental or physiological changes during cell culture, insufficient or inconsistent glycosylation can also be a problem for process consistency.

### SUMMARY OF THE INVENTION

The present invention is based on modification in a eukaryotic cell of the expression of genes which encode enzymes involved in the destruction and/or production of the oligosaccharide portions of glycoproteins. The invention provides a solution to the problem of inconsistencies in and between glycoprotein production lots in addition to decreasing the heterogeneity of glycoforms in the glycoprotein produced. In particular, the modifications to the eukaryotic cell line of the present invention provide that particular genes of interest are or are not functionally expressed leading to more reproducible glycoprotein production. Of particular interest for expression are genes encoding eukaryotic glycosyltransferases and in preferred embodiments the coexpression of at least two glycosyltransferases, especially a galactosyltransferase and a sialyltransferase. Of particular interest among the eukaryotic cells coexpressing particular glycosyltransferases are cells wherein functional expression of a sialidase gene, especially a gene encoding a cytosolic sialidase is reduced or abolished.

According to the present invention, functional gene expression or function may be initiated or augmented or, by contrast, disrupted, by mutation, addition or deletion of one or more genes in the eukaryotic cell line used in the production of glycoproteins. Particular candidate genes for augmentation or addition are the genes encoding mammalian galactosyl- and sialyltransferases. Disruption by, for example mutation, addition or deletion of various sequences containing the genes or fragments thereof, targets enzymes keyed to the destruction or degradation of the oligosaccharide portion of the glycoprotein. Augmentation and addition or disruption may be by any of the methods known to the person skilled in the art, for instance the genes may be inserted or deleted altogether. Gene targeting techniques such as homologous recombination between the genomic gene and a differing but largely homologous nucleic acid sequence introduced into the cells can also be employed to disrupt or augment expression of particular genes. Functional expression may be avoided by, for example, disruption of the gene function by regulation of its transcription or translation, for example, by using antisense technology.

According to a preferred embodiment of the present invention a eukaryotic cell and especially a mammalian cell is modified to functionally express both a galactosyltransferase and sialyltransferase. The expression may be of a gene sequence either homologous or heterologous to the eukaryotic cell. In a further embodiment of the present invention, a cell expressing both a galactosyltransferase and a sialyltransferase is selected so that a sialidase gene is not functionally expressed, the level of functional sialidase produced by the cells being such that sialic acid residues in the carbohydrate side-chains of glycoprotein produced by the cells are not cleaved, or are not cleaved to an extent which affects the function of the glycoprotein.

The eukaryotic cells of the present invention are useful as host cells for the expression of recombinant glycoproteins from nucleic acid introduced into the cells under appropriate conditions. Glycoproteins produced by expression of encoding nucleic acids introduced into these cells have, in preferred embodiments increased galactose and sialic acid content and decreased heterogeneity of carbohydrate and increased uniformity between and within production lots. The cells are especially useful, therefore, for recombinant expression of proteins having sialic acid residues that are necessary for desired enzymatic, immunological, or other biological activity or clearance characteristics of the protein.

A process for producing a heterologous glycoprotein in a eukaryotic cell is also provided. The process includes the steps of introducing into a eukaryotic cell capable of expressing a heterologous glycoprotein at least a first and a second gene capable of being expressed by the eukaryotic cell the first gene comprising a nucleic acid sequence encoding a galactosyltransferase for example a β1,4-galactosyltransferase and the second gene comprising a nucleic acid sequence encoding a sialyltransferase for example an α2,3-sialyltransferase and maintaining the eukaryotic cell under conditions suitable for the expression of the galactosyltransferase and the sialyltransferase as well as the heterologous glycoprotein. Preferred are processes wherein the eukaryotic cell is a Chinese hamster ovary (CHO) cell and the heterologous glycoprotein is a human glycoprotein.

### BRIEF DESCRIPTION OF THE DRAWINGS

FIG. 1A is the negative mode spectra obtained after matrix assisted laser desorption ionization-time of flight (MALDI-TOF) analysis of oligosaccharides isolated from a recombinant tumor necrosis factor receptor-immunoglobulin (TNFR-IgG) chimera purified from cultures using a control CHO cell line. A heterogenous mixture of structures were identified. 2122 represents fully sialylated bi-antennary structures (two branches, 1 fucose, 2 galactose and 2 sialic acid residues); 3133 represents fully sialylated tri-antennary structures; and 4144 represents fully sialylated tetra-antennary structures.

FIG. 1B is the negative mode spectra obtained after MALDI-TOF analysis of oligosaccharides isolated from TNFR-IgG purified from culture of the same CHO cell line transfected with β1,4-galactosyltransferase. The spectra is similar to the spectra obtained on the product of the control cell line (FIG. 1A).

FIG. 2A is the positive mode spectra obtained after MALDI-TOF analysis of oligosaccharides isolated from TNFR-IgG purified from cultures using the control CHO cell line. Structures observed in the positive mode spectra are uncharged residues terminating in galactose on one (G1, 2110) or two (G2, 2120) branches. Residues not containing a terminal galactose (G0, 2100) were also identified.

FIG. 2B is the positive mode spectra obtained after MALDI-TOF analysis of TNFR-IgG oligosaccharides purified from cultures using the CHO cell line transfected with β1,4-galactosyl transferase. The residues present were shifted to the G2 form.

FIG. 3A is the negative mode spectra obtained after MALDI-TOF analysis of oligosaccharides from TNFR-IgG purified from cultures using the control CHO cell line. A heterogenous mixture of structures were identified. 2122 represents fully sialylated bi-antennary structures; 3133 represents fully sialylated triantennary structures; and 4144 represents fully sialylated tetraantennary structures.

FIG. 3B is the negative mode spectra obtained after MALDI-TOF analysis of oligosaccharides isolated from TNFR-IgG purified from culture of CHO cell line transfected with α2,3-sialyltransferase. The heterogeneity observed in the product isolated form the control culture was decreased significantly, resulting in predominantly fully sialylated structures (2122, 3133 and 4144).

FIG. 4A is the positive mode spectra obtained after MALDI-TOF analysis of oligosaccharides isolated from TNFR-IgG purified from cultures using the control CHO cell line. Structures observed in the positive mode spectra are uncharged residues terminating in galactose on one (G1, 2110) or two (G2, 2120) branches. Residues not containing a terminal galactose (G0, 2100) were also identified.

FIG. 4B is the positive mode spectra obtained after MALDI-TOF analysis of TNFR-IgG oligosaccharides purified from cultures using the CHO cell line transfected with α2,3-sialyltransferase.

The spectra is very similar to those seen in the control spectra in the positive mode (FIG. 4A).

FIG. 5A is the negative mode spectra obtained after MALDI-TOF analysis of oligosaccharides isolated from TNFR-IgG purified from cultures using the control CHO cell line. A heterogenous mixture of structures were identified. 2122 represents fully sialylated bi-antennary structures; 3133 represents fully sialylated tri-antennary structures; and 4144 represents fully sialylated tetra-antennary structures.

FIG. 5B is the negative mode spectra obtained after MALDI-TOF analysis of oligosaccharides isolated from TNFR-IgG purified from culture of CHO cell line transfected with both β1,4-galactosyl transferase and α2,3-sialyltransferase. The heterogeneity observed in the product isolated form the control culture was decreased significantly, resulting in predominantly fully sialylated structures (2122, 3133 and 4144) and a decrease in the 2111 glycoform.

FIG. 6A is the positive mode spectra obtained after MALDI-TOF analysis of oligosaccharides isolated from TNFR-IgG purified from cultures using the control CHO cell line. Structures observed in the positive mode spectra are uncharged residues terminating in galactose on one (G1) or two (G2) branches. Residues not containing a terminal galactose (G0) were also identified.

FIG. 6B is the positive mode spectra obtained after MALDI-TOF analysis of TNFR-IgG glycosyl residues purified from cultures using the CHO cell line transfected with both the β1,4-galactosyltransferase and the α2,3-sialyltransferase. The glycosyl residues were shifted to the G1 and G2 form.

FIG. 7A is the negative mode spectra obtained after MALDI-TOF analysis of oligosaccharides isolated from a recombinant tissue plasminogen activator (TNK tPA) purified from cultures using the control CHO cell line. A heterogenous mixture of structures were identified. 2122 represents fully sialylated bi-antennary structures; 3133 represents fully sialylated tri-antennary structures; and 4144 represents fully sialylated tetra-antennary structures.

FIG. 7B is the negative mode spectra obtained after MALDI-TOF analysis of oligosaccharides isolated from TNK purified from culture of CHO cell line transfected with α2,3-sialyltransferase. The heterogeneity observed in the product isolated form the control culture was decreased significantly, resulting in predominantly fully sialylated structures (2122, 3133 and 4144) as for the TNR-IgG product exemplified in FIGS. 3A and 3B.

FIG. 8A is the positive mode spectra obtained after MALDI-TOF analysis of oligosaccharides isolated from TNK purified from cultures using the control CHO cell line. Structures observed in the positive mode spectra are uncharged residues terminating in galactose two (G2) branches. Residues not containing a terminal galactose (G0) were not identified.

FIG. 8B is the positive mode spectra obtained after MALDI-TOF analysis of TNK oligosaccharides purified from cultures using the CHO cell line transfected with α2,3-sialyltransferase. The spectra show a significant decrease in the presence of G2 isoforms and the presence of G0 isoforms not present in the control spectra.

FIG. 9A and 9B show that the immunoreactive sialidase detected in cell extract of the CHO cell population varies (FIG. 9B) more than would be expected based upon assay variation (FIG. 9A).

FIG. 10A depicts sialidase levels in a number of CHO cell clones. The sialidase activity in low (FIG. 10C) and high (FIG. 10B) expressing CHO clones is retained after subcloning.

FIG. 11 depicts sialidase mRNA expression in an example of CHO clones with high and low sialidase expression.

### DETAILED DESCRIPTION OF THE PREFERRED EMBODIMENTS

Definitions

The term eukaryotic cell line is used to refer to cells established in ex vivo culture. It is a characteristic of the eukaryotic cell line of the present invention that it be capable of expressing and secreting large quantities of a particular glycoprotein of interest. Eukaryotic cells used in the production of a desired protein product have the means for glycosylating proteins by addition of oligosaccharide side chains. Such cells, in certain embodiments, also have the capability to remove and/or modify enzymatically part or all of the oligosaccharide side chains of glycoproteins.

A eukaryotic cell line is further manipulated according to the present invention in such a way as to have some genetic modification from the original parent cells capable of expressing the glycoprotein product from which they are derived. Such genetic modification may be the result of introduction of a further nucleic acid sequences as described herein for the regulation of glycosylation of the sought after glycoprotein, or it may be manipulated by the introduction of a gene, possibly with promoter elements, for production within the cells of antisense RNA to regulate expression of another gene. Equally, the genetic modification may be the result of mutation, addition or deletion of one or more nucleotides of a gene or even deletion of a gene altogether, by any mechanism.

Functional expression of a gene refers to production of the protein product encoded by the gene in a form or to the extent required for the product to perform its normal function within the cell environment. Thus, a gene encoding an enzyme involved in protein glycosylation, or deglycosylation, is functionally expressed when enough of the enzyme is produced in a working form to glycosylate, or deglycosylate, at a normal level protein produced in the cell. Functional expression of a gene may be disrupted, initiated or augmented in anyway available to the skilled artisan as for example by transfection or transformation of the eukaryotic host cell. Functional expression of a gene may be disrupted or augmented by, for example, modification of the nucleotide sequence of the gene so that the protein product of the gene is defective in its function or superior in its function. Deletion, addition or other modification of part or all of promoter sequences associated with the gene and involved in transcription of the gene may be employed. Deletion of the gene itself from the genome of the cell, interference with translation of mRNA transcribed from the gene, for example, by interference with antisense RNA, or by any combination of any of these with each other or with any other means known to the person skilled in the art for disrupting, initiating or augmenting gene function may be employed. Such procedures include cloning a particular host cell by limiting dilution and selecting clones based upon functional expression of a particular gene.

The terms “DNA sequence encoding”, “DNA encoding” and “nucleic acid encoding” refer to the order or sequence of deoxyribonucleotides along a strand of deoxyribonucleic acid. The order of these deoxyribonucleotides determines the order of amino acids along the polypeptide chain. The DNA sequence thus codes for the amino acid sequence.

The term “expression vector” refers to a piece of DNA, usually double-stranded, which may have inserted into it a piece of DNA, for example a piece of foreign DNA. Foreign DNA is defined as heterologous DNA, which is DNA not naturally found in the host cell and includes additional copies of genes naturally present in the host genome. The vector is used to transport the foreign or heterologous DNA into a suitable host cell. Once in the host cell, the vector is capable of integration into the host cell chromosomes. The vector contains the necessary elements to select cells containing the integrated DNA as well as elements to promote transcription of polyadenylated messenger RNA (mRNA) from the transfected DNA. Many molecules of the polypeptide encoded by the foreign DNA can thus be rapidly synthesized.

Examples of suitable eukaryotic host cells within the context of the present invention include insect and mammalian cells, and especially mammalian cells such as rodent cells, for example, hamster and murine cells. Examples of such cells include SF9 insect cells (Summers and Smith (1987) Texas Agriculture Experiment Station Bulletin, 1555; and Insect Cell Culture Engineering, Goosen Daugulis and Faulkner Eds. Dekker, New York); Chinese hamster ovary (CHO) cells (Puck et al.., (1958) J. Exp. Med. 108:945-955; Puck (1985) Molecular Cell Genentics, Gottersman M M ed. Wiley Intersciences pp 37-64) including CHO K1 Kao and Puck (1968) Proc. Natl. Acad. Sci. USA 60:1275-1281 (ATCC: CCL61); CHOK1 DUXB11, Urlaub and Chasin, (1980) Proc. Natl. Acad. Sci. USA, 77:4216; dp12.CHO cells (EP 307,247 published Mar. 15, 1989; and DG.44 CHO cells, Urlaub et al., (1986) Somatic Cell Molecular Genentics 12(6):555-566); monkey kidney CV1 line transformed by SV40 (COS-7, ATCC CRL 1651); human embryonic kidney line, (Graham et al., J. Gen Virol., 36:59 [1977]); baby hamster kidney cells (BHK, ATCC CCL 10); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); MRC 5 cells; FS4 cells; and a human hepatoma line (Hep G2). Preferred host cells include Chinese hamster ovary cells deficient in dihydrofolate reductase (DHFR-) (CHO, Urlaub and Chasin, Proc. Natl. Acad. Sci. USA, 77:4216 [1980]); dp12.CHO cells (EP 307,247 published Mar. 15, 1989; and DG.44 CHO cells).

As used herein, “glycoprotein” refers generally to peptides and proteins having more than about ten amino acids and at least one carbohydrate. The glycoproteins may be homologous to the host cell, or preferably, they are heterologous, i.e., foreign, to the host cell being utilized, such as a human protein produced by a Chinese hamster ovary cell. Preferably, mammalian glycoproteins (glycoproteins that were originally derived from a mammalian organism) are used, more preferably, those which are directly secreted into the medium. Examples of mammalian glycoproteins include molecules such as cytokines and their receptors, as well as chimeric proteins comprising cytokines or their receptors, including, for instance tumor necrosis factor alpha and beta, their receptors (TNFR-1; EP 417,563 published Mar. 20, 1991; and TNFR-2, EP 417,014 published Mar. 20, 1991) and their derivatives; renin; a growth hormone, including human growth hormone, and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-12; superoxide dismutase; T-cell receptors; surface membrane proteins; decay accelerating factor; viral antigen such as, for example, a portion of the AIDS envelope; transport proteins; homing receptors; addressins; regulatory proteins; antibodies; chimeric proteins, such as immunoadhesins, (U.S. Pat. Nos. 5,116,964 and 5,565,335) for example, TNFR-IgG (Ashkenazi et al., (1991) Proc. Natl. Acad. Sci. USA 88:1-535-1053, U.S. Pat. No. 5,610,297 and “Ro45-2081 (TNFR55-IgG1) in the Treatment of Patients with Severe Sepsis and Septic Shock: Preliminary Results” Abraham et al., (1995) in Sec. Intern. Autumnal Them. Meeting on Sepsis, Deauville, France); anti-IL-8 (St John et al., (1993), Chest, 103:932 and International Publication No. WO 95/23865); anti-CD11a (Filcher et al., Blood, 77:249-256, Steppe et al., (1991), Transplant Intl. 4:3-7, and Hourmant et al., (1994), Transplantation 58:377-380); anti-IgE (Presta et al., (1993), J. Immunol. 151:2623-2632, and International Publication No. WO 95/19181); anti-HER2 (Carter et al., (1992), Proc. Natl. Acad. Sci. USA, 89:4285-4289, and International Publication No. WO 92/20798); anti-VEGF (Jin Kim et al., (1992) Growth Factors, 7:53-64, and International Publication No. WO 96/30046); and anti-CD20 (Maloney et al., (1994) Blood, 84:2457-2466, Liu et al., (1987) J. Immunol., 130:3521-3526).

“Period of time and under such conditions that cell growth is maximized” and the like, refer to those culture conditions that, for a particular cell line, are determined to be optimal for cell growth and division. Normally, during cell culture cells are cultured in nutrient medium containing the necessary additives generally at about 30-40° C., in a humidified, controlled atmosphere, such that optimal growth is achieved for the particular cell line.

As used herein “culturing for sufficient time to allow amplification to occur” refers to the act of physically culturing the eukaryotic host cells which have been transformed with the DNA construct in cell culture media containing the amplifying agent, until the copy number of the amplifiable gene (and preferably also the copy number of the product gene) in the host cells has increased relative to the transformed cells prior to this culturing.

The term “expression” or “expresses” are used herein to refer to transcription and translation occurring within a host cell. The level of expression of a product gene in a host cell may be determined on the basis of either the amount of corresponding mRNA that is present in the cell or the amount of the protein encoded by the product gene that is produced by the cell. For example, mRNA transcribed from a product gene is desirably quantitated by northern hybridization. Sambrook et al., Molecular Cloning: A Laboratory Manual, pp. 7.3-7.57 (Cold Spring Harbor Laboratory Press, 1989). Protein encoded by a product gene can be quantitated either by assaying for the biological activity of the protein or by employing assays that are independent of such activity, such as western blotting or immunoassay using antibodies that are capable of reacting with the protein. Sambrook et al., Molecular Cloning: A Laboratory Manual, pp. 18.1-18.88 (Cold Spring Harbor Laboratory Press, 1989).

Modes for Carrying out the Invention

The present invention is based upon, among others, the unexpected discovery that for glycoproteins comprising N-linked carbohydrate structures, targeting of sialyltransferases for expression in production host cell lines alone is not uniformly effective in promoting full coverage of N-linked termini with sialic acid. According to the present invention the co-expression of both a galactosyltransferase and a sialyltransferase enzyme that are responsible for the two terminal steps in N-linked oligosacharide biosynthesis in a eukaryotic cell capable of expressing a glycoprotein of interest results in the consistently highest obtainable degree of sialylation and decrease in heterogeneity resulting in increased production lot uniformity regardless of physiological or environmental influences during culture.

According to the invention a eukaryotic cell such as those described above, for example a CHO cell, is modified to functionally express, by any means known in the art, a galactosyltransferase and a sialyltransferase in addition to those which may be expressed as part of the host cell genome. CHO cells are an example of a preferred cell line within the present invention that have been employed for the high yield expression of glycoproteins from engineered vectors. The protein sequence of the glycoprotein expressed by the CHO cell generally comes from DNA transfected into the cell while the structure and extent of the carbohydrate portion of the glycoprotein is determined by the cellular machinery of the host cell, in this example, the CHO cell.

Suitable galactosyltransferases and sialyltransferases for functional expression are those known in the art and are preferably of mammalian origin. According to one aspect of the present invention the host cell line is capable of expressing a human glycoprotein along with human or a combination of human and non-human galactosyl- and sialyltransferases are selected for functional expression in the host cells and processes of the present invention. According to a further aspect of the present invention the processes and cell lines of the present invention employ a sialyltransferase a galactosyltransferase endogenous to the host cell used in the glycoprotein production methods.

For example, when a eukaryotic cell is modified to express both a galactosyltransferase and a sialyltransferase the transferases may be selected based upon the endogenous transferase of the host cell line. In the case of CHO cells it is known that CHO derived recombinant glycoproteins have exclusively α2,3-linked sialic acids, since the CHO genome does not include a gene which codes for a functional α2,6-sialyltransferase. For production of a glycoprotein according to the present invention it is the expression of both a galactosyl and sialyltransferase together and in addition to the natural host cell expression and not the origin of the particular transferase that is important. Options include the expression of endogenous sialyltransferase and endogenous galactosyltransferase. Alternatively, based upon a consideration that human glycoproteins have sialic acid linked in both α2,3- and α2,6-linkages, functional genes for α2,6-sialyltransferases missing from the CHO host genome may be employed.

Nucleic acid encoding the endogenous host cell sequences or the heterologous galactosyltransferases and sialyltransferase genes are available to the skilled artisan and may be obtained by, for example synthesis by in vitro methods or obtained readily from cDNA libraries. The means for synthetic creation of the DNA, either by hand or with an automated apparatus, are generally known to one of ordinary skill in the art. As but one example of the current techniques available for polynucleotide synthesis, one is directed to Maniatis et al., Molecular Cloning—A Laboratory Manual, Cold Spring Harbor Laboratory (1984), and Horvath et al., An Automated DNA Synthesizer Employing Deoxynucleotide 3′-Phosphoramidites, Methods in Enzymology 154:313-326, 1987, hereby specifically incorporated by reference. Alternatively, the gene sequences encoding the sialyltransferases and galactosyltransferase are cloned from cDNA libraries employing techniques available to the skilled artisan. For example polymerase chain reaction techniques may be employed whereby a particular nucleic acid sequence is amplified. Oligonucleotide primers based upon sequences which correspond to the 3′ and 5′ ends of the segment of the DNA to be amplified are hybridized under appropriate conditions and the enzyme Taq polymerase, or equivalent enzyme, is used to synthesize copies of the DNA located between the primers.

Typical examples of DNA sequences which code for sialyltransferases are the sequences for galactoside α2,3-sialyltransferases, and galactoside α2,6-sialyltransferase. The sequences of mammalian sialyltransferases are available from, for example Kitagawa H and Paulson J C (1993) Biochem Biophys Res Comm. 194:375-382 (human); Wen D X, Livingston B D, Medzihradszky K F, Kelm S, Burlingame A L, Paulson J C (1992) J Biol Chem 267(29), 21011-21019 (rat).

Preferred among the galactosyltransferases are the β1,4-galactosyltransferases and preferably the human galactosyltransferases. The sequence of human galactosyltransferases are available in, for example, Masri K A, Appert H E and Fukada M N (1988) Biochem Biophys Res Comm 157, 657-663; Chatterjee S K, Mukerjee S, Tripathi P K (1995) J Biochem Cell Biol 27:329-336; Uejima T, Uemura M, Nozawa S, Narimatsu H (1992) Cancer Res. 52 (22) 6158-6163; Kimura H, Ando T, Uejima T, Nakazato M and Narimatsu H (1994) submitted to DDBJ/EMBL/GenBank databases.

The particular procedure used for the functional coexpression of the transferases is not critical to the invention. For example, any procedure for introducing nucleotide sequences into host cells may be used. These include the use of plasmid vectors, viral vectors, and other methods for introducing genetic material into a host cell. It is necessary that the gene or nucleic acid to be expressed be introduced in such a way that the host cell expresses the enzyme. High level expression is preferred.

For example, expression is typically achieved by introducing into the cells the appropriate transferases along with another gene, commonly referred to as a selectable gene, that encodes a selectable marker. A selectable marker is a protein that is necessary for the growth or survival of a host cell under the particular culture conditions chosen, such as an enzyme that confers resistance to an antibiotic or other drug, or an enzyme that compensates for a metabolic or catabolic defect in the host cell. For example, selectable genes commonly used with eukaryotic cells include the genes for aminoglycoside phosphotransferase (APH), hygromycin phosphotransferase (hyg), dihydrofolate reductase (DHFR), thymidine kinase (tk), neomycin resistance, puromycin resistance, glutamine synthetase, and asparagine synthetase. In selecting an appropriate expression system, a selectable marker for the transferase is chosen to allow for, if necessary, a second transfection with a second suitable amplifiable marker for the expression of the sought after glycoprotein product or to allow additional functional modifications of the host cell. Such modifications may include functional or physical deletion of sialidase or deletion or augmentation of other activities related to improving cell metabolism of the quantity or quality of recombinant product expression.

The level of expression of a gene introduced into a eukaryotic host cell of the invention depends on multiple factors, including gene copy number, efficiency of transcription, messenger RNA (mRNA) processing, stability, and translation efficiency. Accordingly, high level expression of a desired transferase according to the present invention will typically involve optimizing one or more of those factors.

Further, the level of transferase production may be increased by covalently joining the coding sequence of the gene to a “strong” promoter or enhancer that will give high levels of transcription. Promoters and enhancers that interact specifically with proteins in a host cell that are involved in transcription are suitable within the context of the present invention. Among the eukaryotic promoters that have been identified as strong promoters for high-level expression which are preferred within the context of the present invention are the SV40 early promoter, adenovirus major late promoter, mouse metallothionein-I promoter, Rous sarcoma virus long terminal repeat, and human cytomegalovirus immediate early promoter (CMV). Particularly useful in expression of the transferases in addition to the expression of the desired glycoprotein product are strong viral promoters such as the myeloproliferative sarcoma virus (Artel et al., (1988) Gene 68:213-220), SV40 early promoter (McKnight and Tijian (1986) Cell, 46:795-805).

Enhancers that stimulate transcription from a linked a promoter are also useful in the context of the present invention. Unlike promoters, enhancers are active when placed downstream from the transcription initiation site or at considerable distances from the promoter, although in practice enhancers may overlap physically and functionally with promoters. For example, many of the strong promoters listed above also contain strong enhancers (Bendig, (1988) Genetic Engineering, 7:91).

The level of protein production or expression also may be increased by increasing the gene copy number in the host cell.

One method for obtaining high gene copy number is to directly introduce into the host cell multiple copies of the gene, for example, by using a large molar excess of the product gene relative to the selectable gene during cotransfectation.

Kaufman, (1990) Meth. Enzymol., 185:537. With this method, however, only a small proportion of the cotransfected cells will contain the product gene at high copy number. Screening methods typically are required to identify the desired high-copy number transfectants.

The use of splice-donor style vectors such as those described by Lucas et al., (1996) Nuc. Acd Res. 24(9):1774-1779 and WO/9604391 is also preferred.

Yet another method for obtaining high gene copy number involves gene amplification in the host cell. Gene amplification occurs naturally in eukaryotic cells at a relatively low frequency (Schimke,(1988) J. Biol. Chem., 263:5989). However, gene amplification also may be induced, or at least selected for, by exposing host cells to appropriate selective pressure. For example, in many cases it is possible to introduce a transferase gene together with an amplifiable gene into a host cell and subsequently select for amplification of the marker gene by exposing the cotransfected cells to sequentially increasing concentrations of a selective agent. Typically the product gene will be coamplified with the marker gene under such conditions.

The most widely used amplifiable gene for that purpose is a DHFR gene, which encodes a dihydrofolate reductase enzyme. The selection agent used in conjunction with a DHFR gene is methotrexate (Mtx). In this example, a host cell is cotransfected with the transferase gene and a DHFR gene, and transfectants are identified by first culturing the cells in culture medium that contains Mtx. A suitable host cell when a wild-type DHFR gene is used is the Chinese Hamster Ovary (CHO) cell line deficient in DHFR activity, prepared and propagated as described by Urlaub & Chasin, (1980) Proc. Nat. Acad. Sci. USA, 77:4216. The transfected cells then are exposed to successively higher amounts of Mtx. This leads to the synthesis of multiple copies of the DHFR gene, and concomitantly, multiple copies of the transferase gene (Schimke, (1988) J. Biol. Chem., 263:5989; Axel et al., U.S. Pat. No. 4,399,216; Axel et al., U.S. Pat. No. 4,634,665; Kaufman in Genetic Engineering, ed. J. Setlow (Plenum Press, New York), Vol. 9 (1987); Kaufman and Sharp, (182) J. Mol. Biol., 159:601; Ringold et al., J. Mol. Appl. Genet., 1:165-175; Kaufman et al., Mol. Cell Biol., 5:1750-1759; Kaetzel and Nilson, (1988) J. Biol. Chem., 263:6244-6251; Hung et al., (1986) Proc. Natl. Acad. Sci. USA, 83:261-264; Kaufman et al., (1987) EMBO J., 6:87-93; Johnston and Kucey, (1988) Science, 242:1551-1554; Urlaub et al., (1983) Cell, 33:405-412.

Alternatively, host cells may be co-transfected with a transferase gene, a DHFR gene, and a dominant selectable gene, such as a neor gene. (Kim and Wold, (1985) Cell, 42:129; Capon et al., U.S. Pat. No. 4,965,199. Transfectants are identified by first culturing the cells in culture medium containing neomycin (or the related drug G418), and the transfectants so identified then are selected for amplification of the DHFR gene and the product gene by exposure to successively increasing amounts of Mtx.

Another method involves the use of polycistronic mRNA expression vectors containing a product gene at the 5′ end of the transcribed region and a selectable gene at the 3′ end. Because translation of the selectable gene at the 3′ end of the polycistronic mRNA is inefficient, such vectors exhibit preferential translation of the transferase gene and require high levels of polycistronic mRNA to survive selection. Kaufman, (1990) Meth. Enzymol., 185:487; Kaufman, (1990) Meth. Enzymol., 185:537; Kaufman et al., (1987) EMBO J., 6:187. Accordingly, cells expressing high levels of the desired protein product may be obtained in a single step by culturing the initial transfectants in medium containing a selection agent appropriate for use with the particular selectable gene.

A further method suitable within the context of the present invention is integrate the genes encoding the transferases into a transcriptionally active are part of the host cell genome. Such procedures are described in International Application No. PCT/US/04469.

Other mammalian expression vectors such as those that have single transcription units are also useful in the context of the present invention. Retroviral vectors have been constructed (Cepko et al., (1984) Cell, 37:1053-1062) in which a cDNA is inserted between the endogenous Moloney murine leukemia virus (M-MuLV) splice donor and splice acceptor sites which are followed by a neomycin resistance gene. This vector has been used to express a variety of gene products following retroviral infection of several cell types.

Vectors that produce a high level of expression of the gene are particularly useful within the context of the present invention for expressing a transferase that is expressed as part of the host cells endogenous enzyme repertoire. Such expression vector are available to the skilled artisan and include, for example, those described by Lucas et al., (1996) Nuc. Acd Res. 24(9):1774-1779 and WO/9604391 and include those described in the Example sections.

Introduction of the nucleic acids encoding the transferases is accomplished by methods known to those skilled in the art. For mammalian cells without cell walls, the calcium phosphate precipitation method of Graham and van der Eb, (1978) Virology, 52:456-457 may be used. General aspects of mammalian cell host system transformations have been described by Axel in U.S. Pat. No. 4,399,216 issued Aug. 16, 1983. However, other methods for introducing DNA into cells such as by nuclear injection, by protoplast fusion or by electroporation may also be used (Chisholm et al., (1995) DNA Cloning IV: A Practical Approach, Mammalian Systems., Glover and Hanes, eds., pp. 1-41). In the preferred embodiment the DNA is introduced into the host cells using lipofection. See, Andreason, (1993) J. Tiss. Cult. Meth., 15:56-62, for a review of electroporation techniques useful for practicing the instantly claimed invention.

Gene amplification and/or expression may be measured in a sample directly, for example, by conventional Southern blotting, Northern blotting to quantitate the transcription of mRNA (Thomas, (1980) Proc. Natl. Acad. Sci. USA, 77:5201-5205), dot blotting (DNA or RNA analysis), RT-PCR or in situ hybridization, using an appropriately labeled probe, based on the sequences provided herein. Various labels may be employed in constructing probes, most commonly radioisotopes, particularly ³²p However, other techniques may also be employed, such as using biotinmodified nucleotides for introduction into a polynucleotide. The biotin then serves as the site for binding to avidin or antibodies, which may be labeled with a wide variety of labels, such as radionuclides, fluorescens, enzymes, or the like. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

According to the present invention the functional expression of both a galactosyl- and sialyltransferase may be made in a eukaryotic cell line selected for decreased functional expression of a gene encoding enzymes involved in the degradation of carbohydrates. Particular enzymes are sialidases and particularly the sialidases involved in the removal of terminal sialic acid from the carbohydrate moieties of glycoproteins.

For example techniques are available for selecting eukaryotic host cells on the basis of the functional expression of a sialidase. Such procedures include cloning a particular host cell by limiting dilution and selecting clones based upon reduced functional expression of a particular sialidase. Other techniques include, for example, “knock out” or otherwise disrupt the sialidase gene function of a cell line using a technique of homologous recombination. It is also possible to use this approach to disrupt sialidase gene function by targeting the promoter for the gene. A modification which disrupts gene function may be termed a “lesion” and may be an insertion, deletion, replacement or combination thereof, although it is perhaps simplest to use a DNA fragment which has a partial deletion of sialidase encoding sequence. A suitable deletion may be about 50 bp or more. A DNA construct containing the modified gene is introduced into the cell and recombination takes place between the construct and the genomic DNA of the cell.

A marker gene is incorporated in the construct to enable detection of a recombination event. The marker gene may be under the regulatory control of a promoter incorporated in the construct, which may be inducible under suitable conditions. DNA analysis is needed, however, to determine whether recombination is at the correct genomic site. Such DNA analysis may be done by probing for the insert and sequencing regions flanking the insert, thereby determining the presence of sialidase coding sequence in that region, or probing for the sialidase gene and detecting the modification which was made to the insert DNA.

Suitable techniques are described in International Patent Application WO91/01140 and in Hasty et al., Molecular and Cellular Biology, June 1992, 2464-2474, and are known to the person skilled in the art.

Where the target cells are diploid and have two copies of the sialidase gene, the two copies may be disrupted in turn, cells with one mutated copy being expanded and then used in a second stage involving inactivation or other disruption of the second copy of the gene. When no copy is functionally expressed, such cells may be detected by assaying for the absence of activity of the sialidase.

Another technique which may be used in the disruption of functional expression of a sialidase of a cell line, involves antisense RNA. The exact mode of action of antisense RNA in the disruption of normal gene function is not critical to the invention, although it at least partially involves hybridization of the antisense RNA to the complementary mRNA to form double-stranded RNA.

For the culture of the eukaryotic cells expressing the desired protein and modified as described for the instant invention, numerous culture conditions can be used paying particular attention to the host cell being cultured. Suitable culture conditions for eukaryotic cells are well known in the art (J. Immunol. Methods (1983) 56:221-234) or can be easily determined by the skilled artisan (see, for example, Animal Cell Culture: A Practical Approach 2nd Ed., Rickwood, D. and Hames, B. D., eds. Oxford University Press, New York (1992)), and vary according to the particular host cell selected.

The eukaryotic cell culture of the present invention is prepared in a medium suitable for the particular cell being cultured. Commercially available media such as Ham's F10 (Sigma), Minimal Essential Medium ([MEM], Sigma), RPMI-1640 (Sigma), and Dulbecco's Modified Eagle's Medium ([DMEM], Sigma) are exemplary nutrient solutions. In addition, any of the media described in Ham and Wallace,(1979) Meth. Enz., 58:44; Barnes and Sato, (1980) Anal. Biochem., 102:255; U.S. Pat. Nos. 4,767,704; 4,657,866; 4,927,762; 5,122,469 or 4,560,655; International Publication Nos. WO 90/03430; and WO 87/00195; the disclosures of all of which are incorporated herein by reference, may be used as culture media. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range) lipids (such as linoleic or other fatty acids) and their suitable carriers, and glucose or an equivalent energy source. Any other necessary supplements may also be included at appropriate concentrations that would be known to those skilled in the art.

In a particular embodiment, the mammalian host cell is a CHO cell, preferably a DHFR-CHO cell and a suitable medium contains a basal medium component such as a DMEM/HAM F-12 based formulation (for composition of DMEM and HAM F12 media, see culture media formulations in American Type Culture Collection Catalogue of Cell Lines and Hybridomas, Sixth Edition, 1988, pages 346-349) (the formulation of medium as described in U.S. Pat. No. 5,122,469 are particularly appropriate) with modified concentrations of some components such as amino acids, salts, sugar, and vitamins, and optionally containing glycine, hypoxanthine, and thymidine; recombinant human insulin, hydrolyzed peptone, such as Primatone HS or Primatone RL (Sheffield, England), or the equivalent; a cell protective agent, such as Pluronic F68 or the equivalent pluronic polyol; Gentamycin; and trace elements.

For the production of the sought after glycoproteins production by growing the host cells of the present invention under a variety of cell culture conditions is typical. For instance, cell culture procedures for the large or small scale production of proteins are potentially useful within the context of the present invention. Procedures including, but not limited to, a fluidized bed bioreactor, hollow fiber bioreactor, roller bottle culture, or stirred tank bioreactor system may be used, in the later two systems, with or without microcarriers, and operated alternatively in a batch, fed-batch, or continuous mode.

Following the polypeptide production phase, the polypeptide of interest is recovered from the culture medium using techniques which are well established in the art.

The polypeptide of interest preferably is recovered from the culture medium as a secreted polypeotide, although it also may be recovered from host cell lysates.

As a first step, the culture medium or lysate is centrifuged to remove particulate cell debris. The polypeptide thereafter is purified from contaminant soluble proteins and polypeptides, with the following procedures being exemplary of suitable purification procedures: by fractionation on immunoaffinity or ion-exchange columns; ethanol precipitation; reverse phase HPLC; chromatography on silica or on a cationexchange resin such as DEAE; chromatofocusing; SDS-PAGE; ammonium sulfate precipitation; gel filtration using, for example, Sephadex G-75; and protein A Sepharose columns to remove contaminants such as IgG. A protease inhibitor such as phenyl methyl sulfonyl fluoride (PMSF) also may be useful to inhibit proteolytic degradation during purification.

The complex carbohydrate portion of the glycoprotein produced by the processes of the present invention may be readily analyzed if desired, by conventional techniques of carbohydrate analysis to confirm the oligosaccharide content of the glycoprotein. Thus, for example, techniques such as lectin blotting or monosaccharide analysis, well-known in the art, reveal proportions of terminal mannose, N-acetylglucosamine, sialic acid or other sugars such as galactose. Termination of mono-, bi-, tri-, or tetra-antennary oligosaccharide by sialic acids can be confirmed by release of sugars from the protein using anhydrous hydrazine or enzymatic methods and fractionation of oligosaccharides by ion-exchange or size exclusion chromatography, mass spectrometry or other methods well-known in the art. The pI of the glycoprotein can also be measured, before and after treatment with neuraminidase to remove sialic acids. An increase in pI following neuraminidase treatment indicates the presence of sialic acids on the glycoprotein.

The carbohydrate structures of the present invention generally occur on the protein expressed as N-linked carbohydrates, although O-linked structures, when present, would also be modified. N-linked glycosylation refers to the attachment of the carbohydrate moiety via GlcNAc to an asparagine residue in the peptide chain. The N-linked carbohydrates all contain a common Man1-6(Man1-3)Manβ1-4GlcNAcβ1-4GlcNAcβ-R core structure. Therefore, in the core structure described, R represents an asparagine residue of the produced protein. The peptide sequence of the protein produced will contain an asparagine-X-serine, asparagine-X-threonine, and asparagine-X-cysteine, wherein X is any amino acid except proline. O-linked carbohydrates, by contrast are characterized by a common core structure, which is the GalNAc attached to the hydroxyl group of a threonine or serine. Of the N-linked, the most important are the complex N- carbohydrates. Such complex carbohydrates will contain several antenanary structures. The mono-, bi-, tri,-, and tetra-, outer structures are important for the addition of terminal sialic acids. Such outer chain structures provide for additional sites for the specific sugars and linkages that comprise the carbohydrates of the instant invention.

The resulting carbohydrates can be analyzed by any method known in the art including those methods described herein. Several methods are known in the art for glycosylation analysis and are useful in the context of the present invention. Such methods provide information regarding the identity and the composition of the oligosaccharide attached to the peptide. Methods for carbohydrate analysis useful in the present invention include but are not limited to capillary electrophoresis (Rassi and Nashabeh, High Performance Capillary Electrophoresis of Carbohydrates and Glycoconjugates, In, Carbohydrate Analysis (Z. El Rassi, ed.) (1995) 58:267-360), fluorophore-assisted carbohydrate electrophoreses (Starr et al., (1996) J. Chrom. A 720:295-321), high pH anion exchange chromatography pulsed amperometric detection (HPAEC PAD) (Lee (1996) J. Chrom. A 720:137-151), matrix assisted laser desorption/ionization time of fight mass spectrometry (Harvey (1996) J. Chrom. A 720:429-447; Papac et al., (1996) Anal. Chem. 68:3215-3223; Field et al. (1996) Anal. Biochem. 239:92-98; Hooker et al., (1995) Biotechnology and Bioeng. 48:639-648), high performance liquid chromatogrphy (El Rassi (1995) Reversed phase and hydrophobic interaction chromatogrphy of carbohydrates and glycoconjugates. In, Carbohydrate Analysis (Z. El Rassi, ed.) 58:267-360), and electrospray ionization mass spectrometry Roberts G. D. (1995) Anal. Chem. 67:3613-3625).

Additionally, methods for releasing oligosaccharides are known. These methods include 1)enzymatic, which is commonly performed using peptide-N-glycosidase F/endo-β-galactosidase; 2) β elimination using harsh alkaline environment to release mainly O-linked structures; and 3) chemical methods using anhydrous hydrazine to release both N-and O-linked oligosaccharides

Analysis can be performed using the following steps:

1. Dialysis of the sample against deionized water, to remove all buffer salts, followed by lyophilization.

2. Release of intact oligosaccharide chains with anhydrous hydrazine.

3. Treatment of the intact oligosaccharide chains with anhydrous methanolic HCl to liberate individual monosaccharides as O-methyl derivative.

4. N-acetylation of any primary amino groups.

5. Derivatization to give per-O-trimethylsilyl methyl glycosides.

6. Separation of these derivative, by capillary GLC (gas-liquid chromatography) on a CP-SIL8 column.

7. Identification of individual glycoside derivatives by retention time from the GLC and mass spectroscopy, compared to known standards.

8. Quantitation of individual derivatives by FID with an internal standard (13-O-methyl-D-glucose).

Neutral and amino-sugars can be determined by high performance anion-exchange chromatography combined with pulsed amperometric detection (HPAE-PAD Carbohydrate System, Dionex Corp.). For instance, sugars can be released by hydrolysis in 20% (v/v) trifluoroacetic acid at 100° C. for 6 h. Hydrolysates are then dried by lyophilization or with a Speed-Vac (Savant Instruments). Residues are then dissolved in 1% sodium acetate trihydrate solution and analyzed on a HPLC-AS6 column as described by Anumula et al. (Anal. Biochem. 195:269-280 (1991).

Sialic acid can be determined separately by the direct calorimetric method of Yao et al. (Anal Biochem. 179:332-335 (1989)) in triplicate samples. In a preferred embodiment the thiobarbaturic acid (TBA) of Warren, L. J. Biol Chem 238:(8) (1959) is used.

Alternatively, immunoblot carbohydrate analysis may be performed. According to this procedure protein-bound carbohydrates are detected using a commercial glycan detection system (Boehringer) which is based on the oxidative immunoblot procedure described by Haselbeck and Hosel (Haselbeck et al. (1990) Glycoconjugate J., 7:63). The staining protocol recommended by the manufacturer is followed except that the protein is transferred to a polyvinylidene difluoride membrane instead of nitrocellulose membrane and the blocking buffers contained 5% bovine serum albumin in 10 mM tris buffer. pH 7.4 with 0.9% sodium chloride. Detection is made with antidigoxigenin antibodies linked with an alkaline phosphate conjugate (Boehringer), 1:1000 dilution in tris buffered saline using the phosphatase substrates, 4-nitroblue tetrazolium chloride, 0.03% (w/v) and 5-bromo-4 chloro-3-indoyl-phosphate 0.03% (w/v) in 100 mM tris buffer, pH 9.5, containing 100 mM sodium chloride and 50 mM magnesium chloride. The protein bands containing carbohydrate are usually visualized in about 10 to 15 min.

The carbohydrate may also be analyzed by digestion with peptide-N-glycosidase F. According to this procedure the residue is suspended in 14 μl of a buffer containing 0.18% SDS, 18 mM beta-mercaptoethanol, 90 mM phosphate, 3.6 mM EDTA, at pH 8.6, and heated at 100° C. for 3 min. After cooling to room temperature, the sample is divided into two equal parts. One aliquot is not treated further and serves as a control. The econd fraction is adjusted to about 1% NP-40 detergent followed by 0.2 units of peptide-N-glycosidase F (Boehringer). Both samples are warmed at 37° C. for 2 hr and then analyzed by SDS-polyacrylamide gel electrophoresis.

The following examples are provided to illustrate the invention only, and should not be construed as limiting the scope of the invention. All literature citations herein are expressly incorporated by reference.

### EXAMPLES

### Example I

Isolated Sequence

Based on published sequences (Wen D X, Livingston B D, Medzihradszky K F, Kelm S, Burlingame A L, Paulsen J C (1992) J Biol Chem 267(29), 21011-21019; Masri K A, Appert H E and Fukada M N (1988) Biochem Biophys Res Comm 157, 657-663), full length cDNA for human α2,3 sialyltransferase gene and the human β1,4 galactosyltransferase gene using PCR were isolated. The sequence of the sialyltransferase was confirmed to be identical to published sequences. The sequence of the galactosyltransferase gene had several base changes from any of 7 published sequences (supra). There were no difference in these proteins at the amino acid level.

Constructed Unique Expression Vectors:

The vectors used for overexpression were a version of an expression plasmid pSVI.ID.LL described in Lucas et al., (1996 Nuc. Acid Res. 24(9):1774-1779 which replaces the DHFR selectable marker with puromycin resistance sequences and the SV40 promoter/enhancer with a promoter derived from the myoproliferative sarcoma virus (MPSV). The characteristic of this system that is important is that the stronger promoter (MPSV) provides very high levels of gene expression, without amplification, after selection with puromycin. This vector allowed us to construct either host or product cell lines that overexpress the glycosyltransferases without using the DHFR system that can be reserved for product expression. The DP-12 CHO host cells are already neomycin resistant due to previous transfection of the proinsulin gene with this selectable marker (EP 07 247).

Analysis of Glycosyltransferase Expression and Effects on Product Quality:

Overexpression of human α2,3-sialylt-ansferase and β1,4 galactosyltransferase were confirmed by northern analysis as well as by measurement of intracellular sialyltransferase and galactosyltransferase activity. The content of sialic acid on recombinant protein was measured using chromatographic (Anumula, R. K. (1995) Anal. Biochem., 230:24-30) analysis and the structure and quantity of oligosaccharides on the purified molecules were evaluated using MALDI-TOF mass spectrometry (Kaufman (1995) J. Biotech. 41: 155-175; James (1996) Cytotech 22:17-24).

Results

We superfected recombinant cell lines producing TNFR-IgG (Jin et al., J. Infect dis. 1994 170:1323-1326) and TNK tPA (Benedict et al., (1995) Circulation 92:3032-3040) with the MPSV vector containing α2,3 sialyltransferase or β1,4 galactosyltransferase separately or in combination. High level expression of the transferase genes in pools of puromycin resistant cells was verified by mRNA analysis and by enzyme activity assay.

Control and superfected cell lines were cultured under identical conditions in 2L bioreactors (n=2 per cell line). The conditions chosen were similar to those used for large scale TNFR-IgG and TNK production processes. For TNFR-IgG, culture conditions for low specific productivity (International Publication Number WO 96/39488) were employed (see figures) along with conditions which normally yield a product with diminished sialic acid content. In the later case there was no decrement in TNFR-IgG quality noted when product was expressed from sialyltransferase expressing host cell lines.

Product produced from these cell lines was purified and evaluated for the content of terminal sialic acid and for the overall structure of the N-linked glycans using MALDI-TOF mass spectroscopy. In addition the clearance properties of the engineered cell lines were compared in rabbits to wild-type TNK and TNK that had been subjected to in vitro remodeling processes.

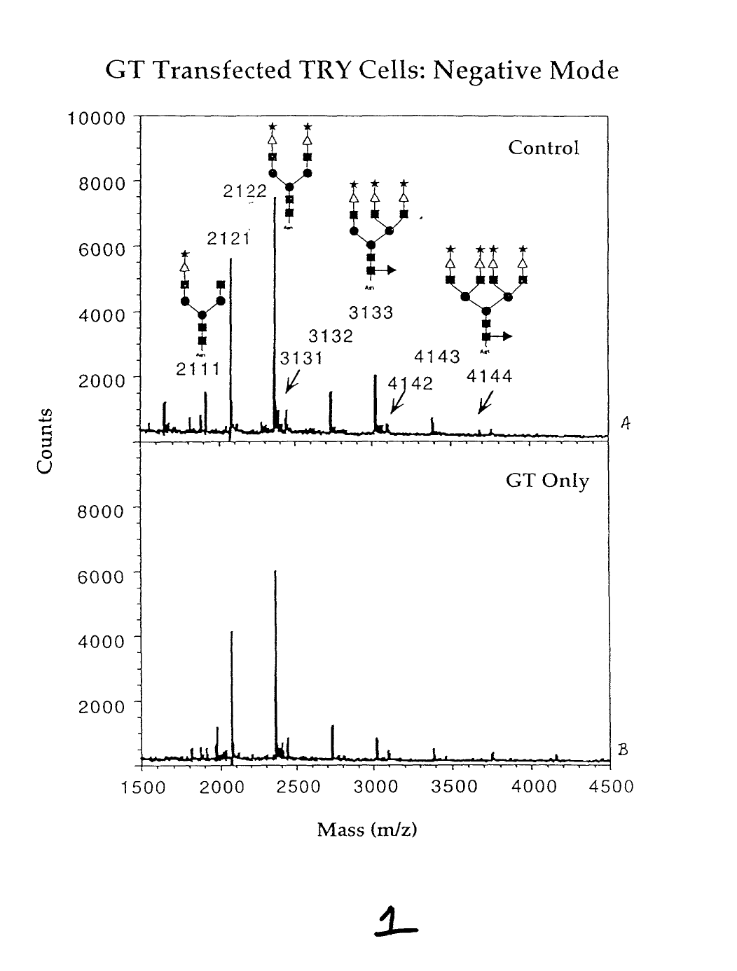

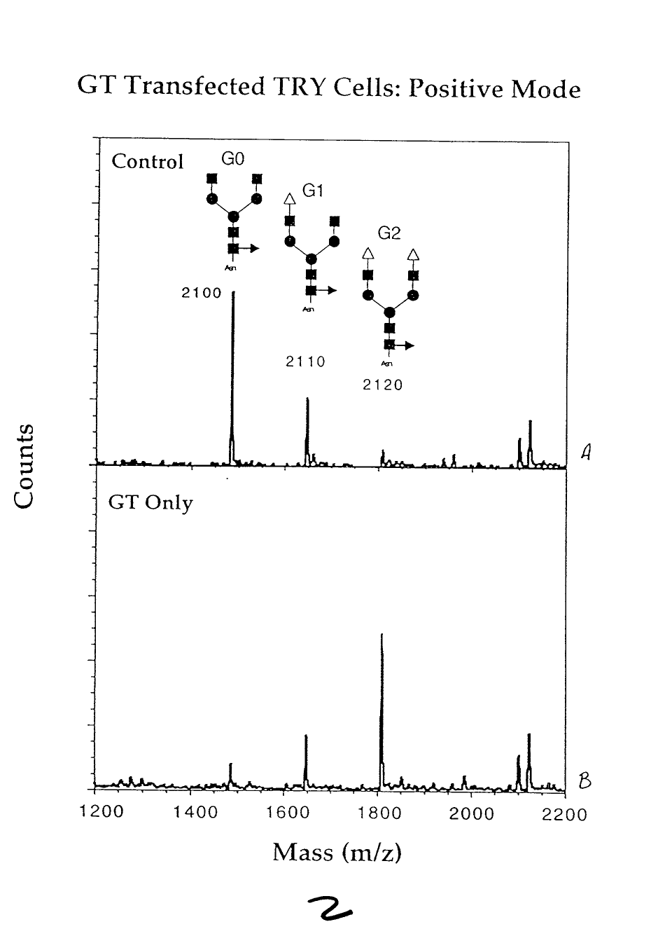

The negative mode spectra obtained after MALDI-TOF analysis of TNFR-IgG purified from cultures using the control line are shown in FIG. 1A. Only charged, sialic acid-containing oligosaccharides are detected in the negative mode. A heterogeneous mixture of structures is evident. Fully sialylated bi-, tri- and tetra-antennary structures are present (2122, 3133 and 4144) along with structures missing sialic acid on one or more branches and a single structure missing both terminal galactose and sialic acid on one branch (2111). The positive mode spectrum from the TNFR-IgG purified from non-transfected cultures is shown in FIG. 2A. Wild-type structures observed in the positive mode are uncharged, terminating in galactose on one or two branches (G1, G2) or with GlcNAc alone (G0). For TNFR-IgG these uncharged structures are thought to be mainly associated with the IgG portion of the molecule and are not subject to sialylation. These data show that the majority of oligosaccharides monitored in the positive mode for TNFR-IgG are non-galactosylated, although small amounts of G1 and G2 structures can be seen.

In the negative mode, TNFR-IgG produced concurrently with overexpression of β1,4-galactosyltransferase was similar to that produced by control cells (FIG. 1B). This result was expected since most of the heterogeneity observed in the negative mode results from undersialylation. However, the uncharged oligosaccharides present on TNFR-IgG co-expressed with β1,4-galactosyltransferase were now shifted towards more highly galactosylated, G2 forms (FIG. 2B).

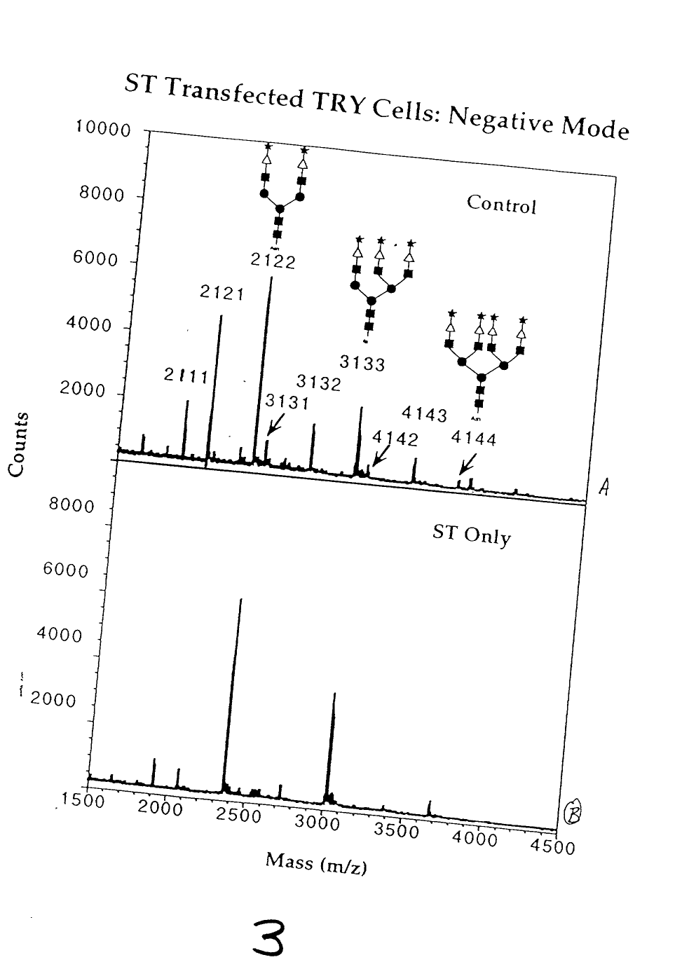

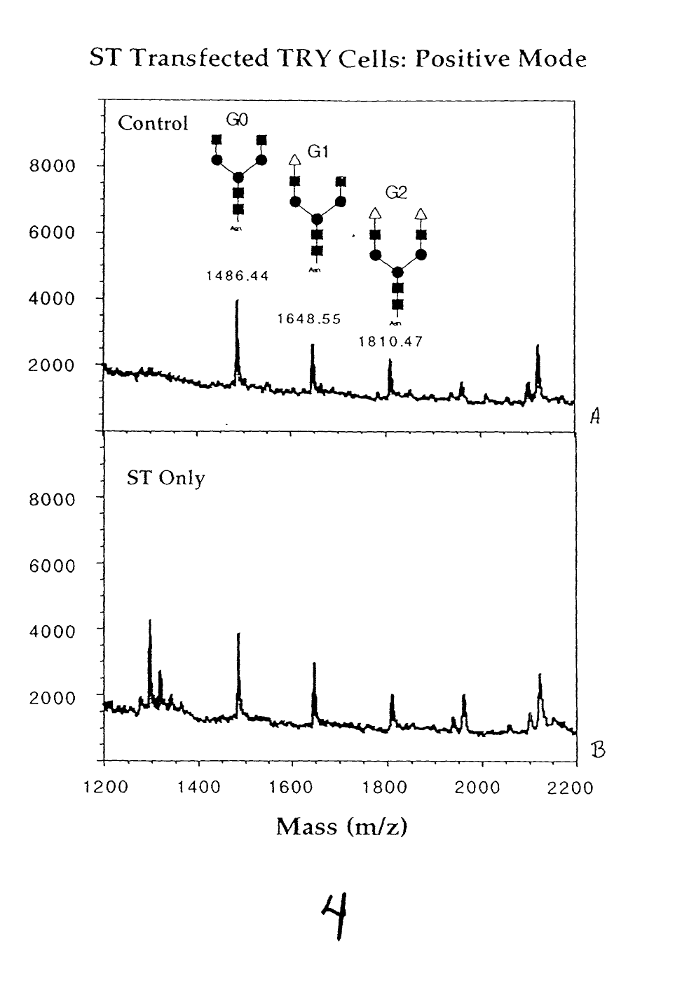

In contrast, when TNFR-IgG was expressed by cells concurrently overexpressing the α2,3 sialyltransferase, the predominant effect was noted in the negative mode MALDI spectra (FIG. 3A vs 3B). The heterogeneity observed in the product isolated from the control culture was decreased significantly, resulting in predominately fully sialylated structures, with only minor amounts of under sialylated material remaining (2121 and 2111). The oligosaccharide structures of TNFR-IgG purified from cultures of cells overexpressing the α2,3 sialyltransferase were very similar to those seen in the control spectrum in the positive mode (FIG. 4A vs 4B).

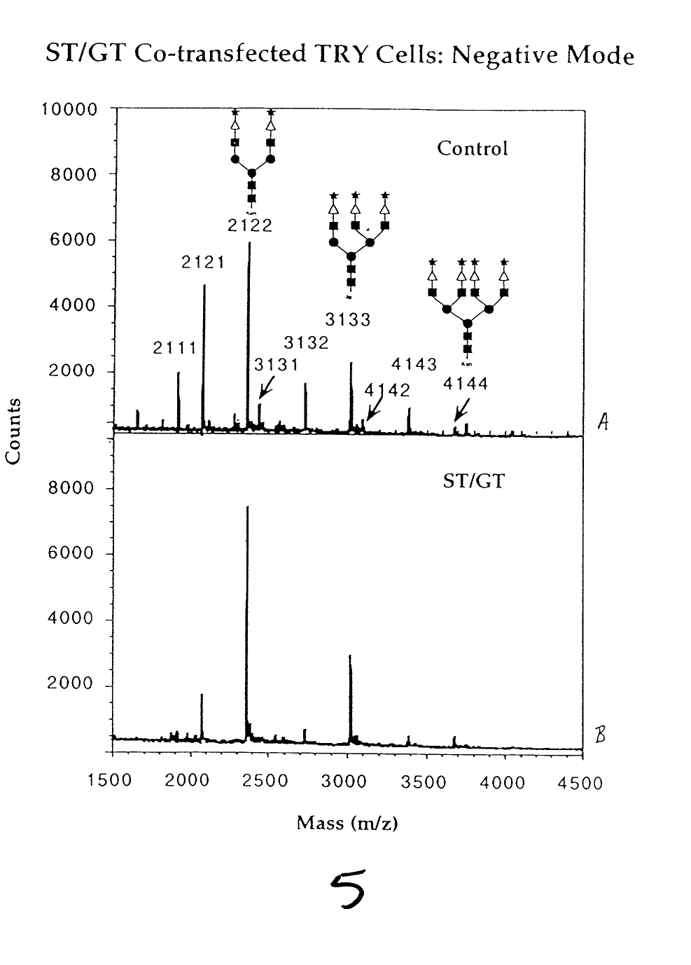

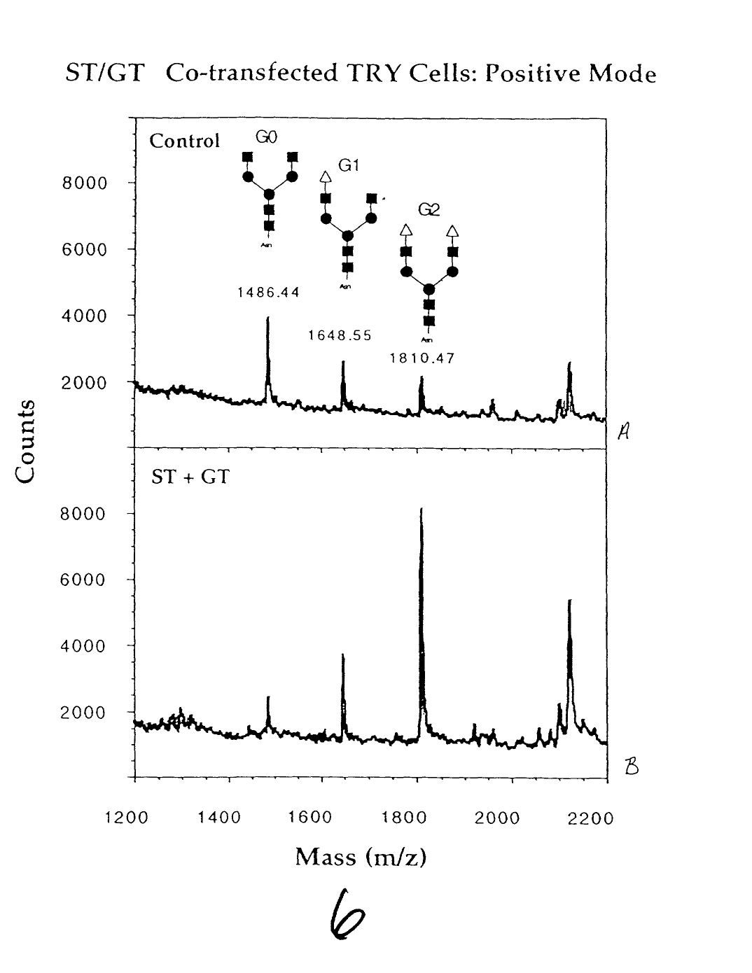

The effect of co-expression of both glycosyltransferases on the quality of TNFR-IgG was examined. A decrease in heterogeneity, comparable to that seen in the α2,3-sialyltransferase only cases (but including a decrease in the presence of 2111-type structures), was again observed in the negative mode spectrum (FIGS. 5A and 5B). Neutral oligosaccharides on TNFR-IgG produced by these same cultures was found to be predominately G1 and G2 (FIGS. 6A and 6B).

### Example II

TNK Cells Overexpressing α2,3 sialyltransferase

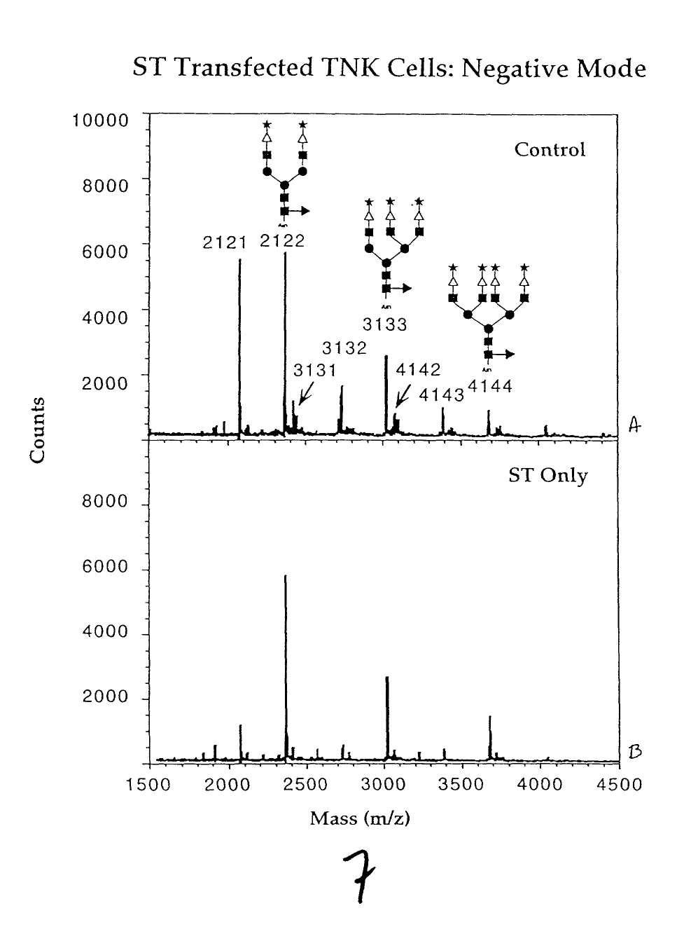

In order to address whether overexpression of glycosyltransferases could alter the structure of the N-linked oligosaccharides on other recombinant proteins, cells expressing TNK tPA were also superfected with α2,3-sialyltransferase. Purified TNK tPA from non-transfected cells showed markedly increased heterogeneity in the content of terminal sialic acid residues compared to material purified from α2,3 sialyltransferase over-expressing cell lines (FIG. 7A vs 7B). As for the TNFR-IgG materials examined, TNK tPA expressed concurrently with high levels of α2,3 sialyltransferase showed predominately fully sialylated bi- and tri-antennary forms, with a minimal amount of 2121 structures remaining.

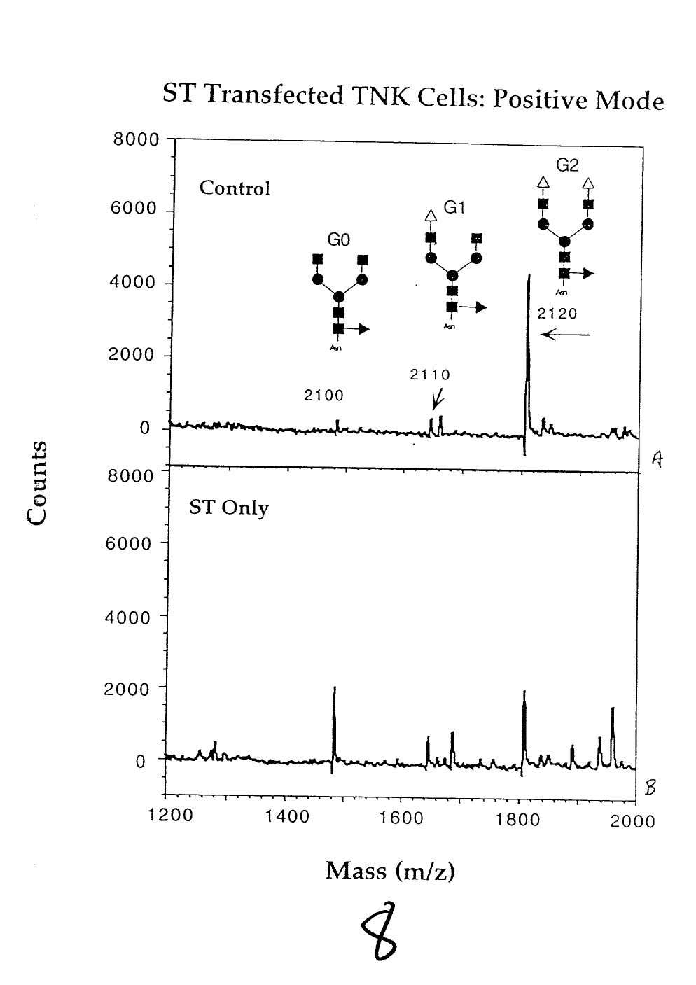

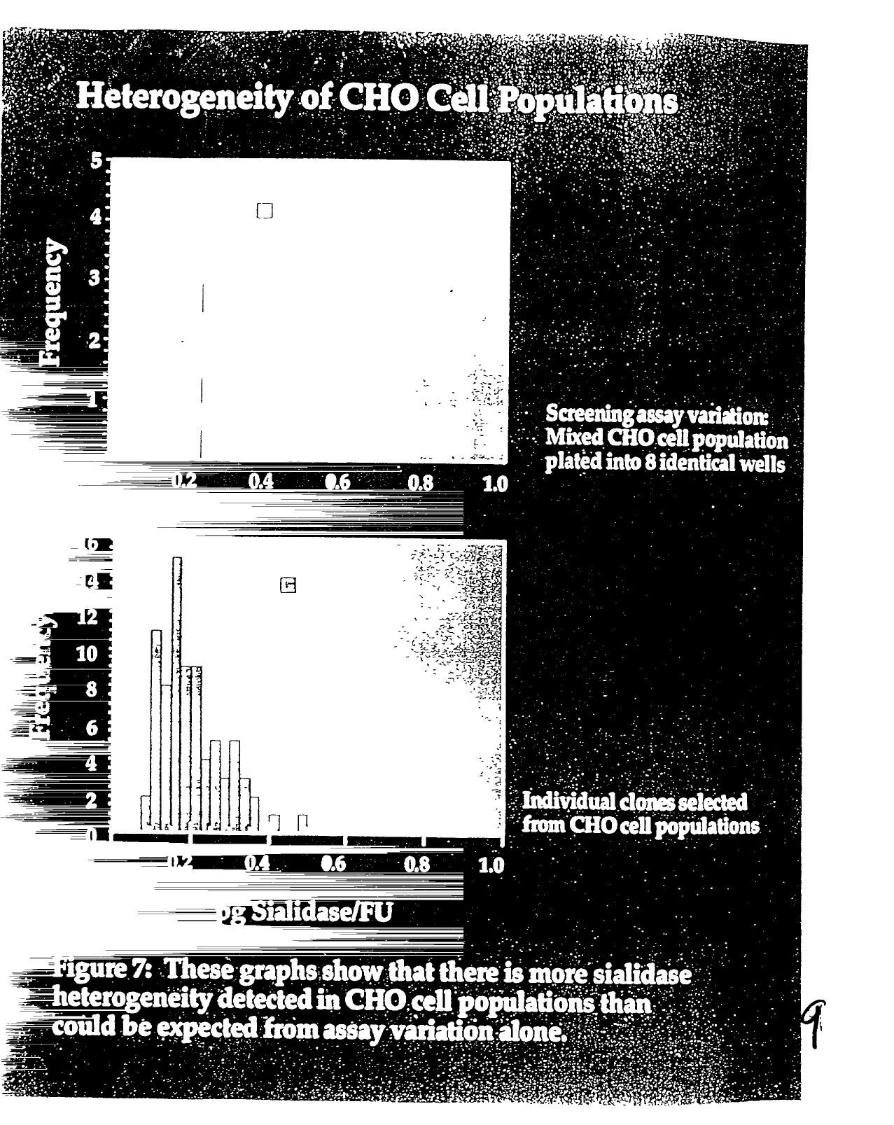

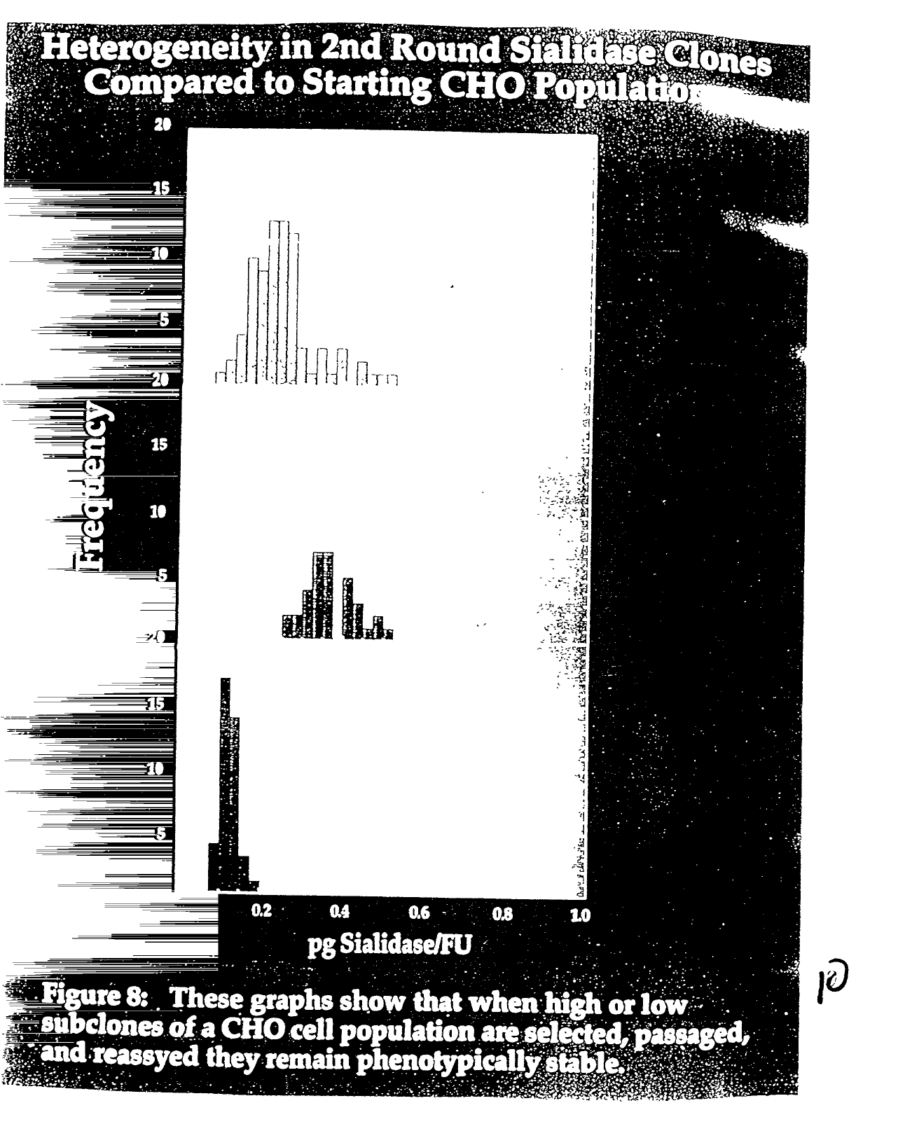

The positive mode spectra for TNK tPA produced from α2,3 sialyltransferase cell lines is shown in FIG. 8. In the control material, predominately G2 structures were identified. The occurrence of these structures was reduced in material from the α2,3 sialyltransferase cells, since sialylation moves these structures into the negative mode. However, the spectra from the transfected material also unexpectedly showed additional presence of G0 structures which were not present in the control spectra. These structures may be the result of b-galactosidase activity released into the supernatant of this culture or may arise as a result of fermentation to fermentation variation in the type of oligosaccharide structures synthesized. Since sialyltransferase cannot act on structures which do not have terminal galactose, the overexpression of the sialyltransferase gene in these cells is insufficient to avert the generation of this heterogeneity. These data illustrate the importance of controlling both galactosylation and sialylation to arrive at consistent processes. Experiments are presently underway to examine the effects overexpression of both sialyltransferase and galactosyltransferase on TNK tPA product quality.

### Example III

Development of a Specific Sialidase ELISA & Identification of CHO Cells with Naturally Occurring Low Sialidase Expression:

A polyclonal antibody directed against CHO sialidase was used to construct a sandwich ELISA specific for the CHO cell enzyme. This assay uses recombinant CHO sialidase, expressed in bacculovirus as the standard and is capable of measuring sialidase in extracts of CHO DP.12 cells cultured at 96-well scale. We used this assay to characterize the heterogeneity of sialidase expression in a population of CHO cells which is used as the parent cell line for transfection. Briefly, cells from this line were plated at clonal dilution in 96-well plates. Clones growing out of the initial plating were then characterized for sialidase specific activity (sialidase/cell). Clones exhibiting low or high specific activity of sialidase were expanded, retested in the same format and re-cloned in order to confirm the stability of the low or high sialidase phenotype. Confirmation of the sialidase phenotype of the resulting cell lines was performed at the mRNA level using northern analysis.

Selection of a CHO Cell Host with a Naturally Occurring Low Sialidase Expression Level:

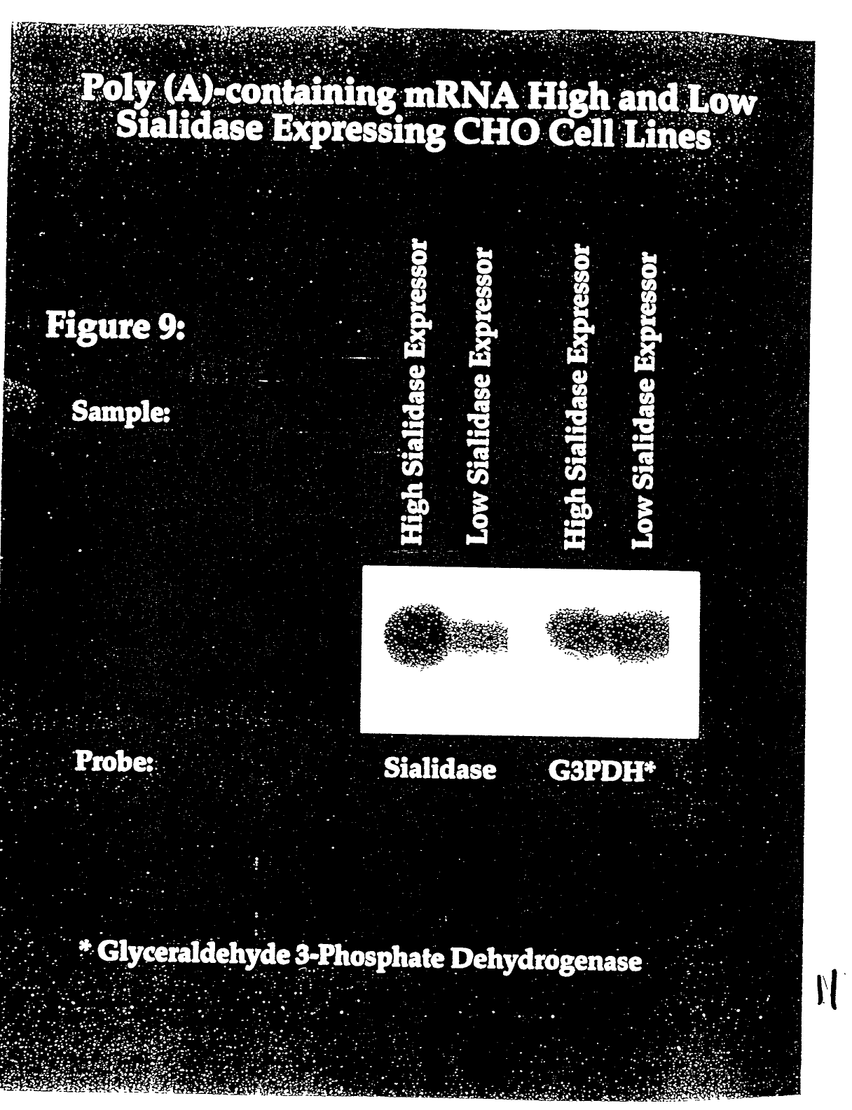

A series of 78 clones resulting from dilution cloning of our starting cell population was examined for their specific content (enzyme/cell) of sialidase. The content of enzyme among the clones was found to vary more (CV 40%) than would be expected due to assay variation alone (12.6%, FIG. 11). High and low clones from these initial screens were selected and subjected to two additional rounds of subcloning. Subclones derived from the second round of subcloning were re-assayed for their specific sialidase content and shown to retain the low or high sialidase phenotype (FIG. 12). Moreover, the heterogeneity among the subclones was reduced to a level (15-16%) not significantly greater than that of the assay alone, suggesting that all cells from the selected cell line were homogeneous in terms of their sialidase expression. Low and high expressing sialidase clones were also analyzed for sialidase mRNA expression. Results shown in FIG. 13 confirm that the difference in the content of enzyme/cell between the high and low clones results from altered expression of sialidase mRNA. The molecular basis for differential expression of sialidase in our starting cell population is not clear at this time.

Thus by screening of the parental CHO cell we have generated CHO cell lines which we believe to be phenotypically low in sialidase expression.

### Example IV

### CONSTRUCTION OF AN ANTISENSE CELL LINE

Methods

1)Sialidase antisense constructs—The sialidase cDNA was previously cloned and isolated from a CHO cell cDNA library (Ferrari et al., (1994) Glycobiology 4:9188-9192). Antisense plasmids were constructed using the entire 1.4 kb sialidase cDNA along with several smaller fragments that were generated by EcoR I and Pst I digestion of the full length CDNA. These included a 189 bp 5′ untranslated portion of the cDNA, a 474 bp 5′ coding region and a 686 bp 3′ coding segment. All fragments including the full length cDNA were inserted in the inverted orientation into an SV40 early promoter expression vector. The full length cDNA was also inserted in the sense orientation and used a control.

2)Transfection of CHO cells with antisense plasmids and a puromycin resistance marker—Preparations of antisense plasmids (10 mg) were electroporated into the parent CHO cell line (DP12, DHFR-) using both linear (HPA I treated) and non-linear constructs. Cotransfections with a plasmid conferring puromycin resistance (Clontech, Inc.) as a selectable marker were carried out at 1:2 and 1:20 ratios of the puromycin plasmid to antisense vector. As a control, the parent cell line was transfected with the puromycin plasmid alone.

The transfected cells were cultured in a monolayer in high glucose-MEM medium supplemented with 5% fetal calf serum and 1 mM GHT. After 24 hours, puromycin was added to a working concentration of 10 mg per ml. After 10 to 14 days single colonies were selected and grown in duplicate, in 100 mm petri dishes.

3)Screening transfectants for sialidase activity—Sialidase assays were carried out on confluent monolayer cultures of the puromycin resistant colonies. Cells were harvested with trypsin and washed with phosphate buffered saline (PBS). Cells were disrupted by the addition of 0.2% (w/v) saponin in water and freeze-thawed one time. Sialidase activity in the homogenates was determined using 4-methylumbelliferyl-N-acetyl neuraminic acid as substrate (Warner et al., (1993) Glycobiology, 3:455-463). Triplicate assays were carried out on each sample and protein levels were determined using the BCA protein reagent kit (Pierce Chem. Co.).

After an initial screening, clones with sialidase activity reduced by 40% or greater were passaged a second time and reassayed. In several cases, some clones with low sialidase in the initial round of screening had near normal enzyme activity after one passage. These clones were not characterized further. The remaining low expressors were passaged a third time and rescreened for sialidase. The cell line with the lowest sialidase activity, ˜40% of the wildtype control, contained the 474 bp antisense segment (clone 474) of the sialidase cDNA corresponding to the 5′ coding region of the sialidase gene.

4)Identifying insertion of antisense construct into CHO cell genome—After stable antisense-transfected clones were obtained, verification that the antisense DNA was incorporated into the CHO cell genome was made using the polymerase chain reaction (PCR) with CHO cell genomic DNA as template and nucleotide primers corresponding to nucleotide sequences of the SV 40 expression vector spanning the antisense insert. Primers were designed to give 722 bp PCR product for the clone containing the 474 bp antisense insert. The PCR reaction conditions were 1 cycle at 940 C. for 2.5 min, 35 cycles at 1 min at 940 C., 2 min at 540 C. and 2 min at 720 C. and 1 cycle at 720 C. for 7 min. The reaction mixtures were resolved on a 6% polyacrylamide gel stained with ethidium bromide.

5)Transfection of antisense clone 474 with a plasmid encoding a human Dnase A glycoprotein—The 474 antisense cell line was transfected with an SV40 based expression plasmid containing both the human glycoprotein (Skak et al., (1990) Proc. Natl. Acad. Sci. 87:9188-9192) and dihydrofolate reductase (DHFR). Transfections were carried out using LipofectAMINE (Gibco BRL). Transfectants were screened using a monoclonal antibody based ELISA assay for DNase expression and enzyme assay for sialidase activity. One selected clone was subcloned further into two additional clones (9B and 5B). The D12 wild-type control line was also transfected with the plasmid containing the human glycoprotein and developed with the similar procedure. All cell lines were adapted to grow in suspension cultures.

7)Determination of sialidase levels in cell culture fluid—The levels of sialidase in the cell culture fluid of the fermentor cultures of wild-type and antisense expressing CHO cell lines was quantified on a daily basis. The culture fluid was subjected to centrifugation (48,000×G, 1 hr, 40 C.) to remove cell debris and the resulting supernatant (4.0 ml) was concentrated about 10 fold using a Microsep concentrator with a 10 kDa exclusion limit (Pall Filtron Corp). The sialidase levels in the concentrated fluid were determined by enzyme assay using the fluorometric substrate.

8)Purification of the human glycoprotein from cell culture fluid—DNase in the cell culture fluid from the 474/DNase and D12/DNase cell cultures was isolated and purified using a modification of a protocol described in Cacia et al., (1993) J. Chromatography 634:229-239). The fluid was concentrated and the buffer exchanged using a Filtron ultrasette or miniultrasette diafiltration apparatus (Filtron, Inc.) with 0.025 M Hepes buffer containing 1 mM CaC12, pH 7.0 at a conductivity of 0.6 mmho. The concentrated fluid was subjected to DEAE column chromatography (3 cm3 total column volume). After application of the protein, the column was washed with a buffer containing 10 mM sodium acetate, pH 4.5 and 43 mM NaCl. The glycoprotein was eluted with a buffer containing 10 mM sodium acetate, pH 4.5, 10 mM CaCl₂, 53 mM NaCl. The fractions containing enzyme activity were pooled and subjected to cation exchange chromatography using a Lichrosphere SO3—tentacle cation exchange column (EM Separations, Inc). The column was equilibrated in 10 mM sodium acetate, pH 4.5, 1 mM CaCl2. The protein was eluted using a linear gradient of increasing amounts of the equilibration buffer containing 1 M NaCl. Typically about 7 μg of protein/ml of fluid were obtained.

### RESULTS

The segment of the sialidase antisense gene that gave maximal reduction of enzyme activity was determined empirically by evaluating several small DNA fragments along with the full length cDNA in antisense expression vectors (Helene and Toulme (1990) Biochem. Biophys. Acta 1049:99-125; Takayman and Inouye (1990) Biochem. Mol. Biol. 25:155-148). Constructs were made with the 189 bp 5′ noncoding region, a 474 bp and a 686 bp segment of the 5′ and 3′ coding regions, respectively. Each antisense construct was co-transfected into CHO cell along with a plasmid encoding the puromycin resistance gene. About 10-12 clones (for a total of about 45 clones) from the puromycin resistant pools were subsequently screened for sialidase activity and the activity compared with the D12 parental cell line. Only those cell lines that consistently retained low sialidase levels upon expansion of the culture through two passages were studied further. We consistently observed higher enzyme levels in the D12 wild-type cell line transfected with the vector conferring puromycin resistance alone. As expected, transfectants that contained the full length sialidase cDNA in the sense orientation also had higher enzyme levels than the wild-type D12 cells. From the 45 antisense clones screened for sialidase activity, one clone, 474 #16, was identified that consistently gave about 40 % residual sialidase activity after several passages. This low sialidase expressing clone was obtained using the 474 bp 5′ antisense coding segment of the sialidase gene.

In order to determine if the 474 bp antisense segment of the sialidase gene was present in the CHO cell genome, a PCR (polymerase chain reaction) assay was carried out using genomic DNA from clone 474#16 as a template and vector-based PCR primers spanning the 474 bp antisense insert. No PCR reaction product of the anticipated size was detected using template DNA from the wild-type cell line transfected with the puromycin vector alone, D12/pur, or with genomic material from the wild-type host cell line, D12. Also, no product was observed in the absence of template. Using DNA from the antisense clone 474#16 as template, a 722 bp PCR reaction product was observed, as expected, verifying that the 474 bp antisense DNA segment had integrated into the CHO cell genome along with segments of the expression vector. The PCR reaction product is larger than the 474 bp antisense insert because the vector-based primers include 248 bp of vector nucleotide sequence. Some of the puromycin resistant antisense clones with moderately reduced sialidase activity, ˜65-70% residual activity relative to wild-type, did not give a PCR product when analyzed with this method. In these clones, it is likely that integration of the puromycin vector but not the antisense vector had occurred. These cells may be naturally occurring low sialidase expressors. However, the sialidase levels in these or any other clones tested were not as low as that found in clone 474#16 .

In order to test if the reduction in sialidase levels in clone 474#16 was sufficient to give an improvement in the sialic acid content of a model glycoprotein produced in a batch culture setting, the cell line was used as a host for the expression of human glycoprotein.

Although sialidase activity was not completely eliminated in the antisense cell lines, the 60% reduction in activity resulted in a 20-37% increase in sialic acid content (about 0.6-1.1 mole of additional sialic acid out of a total of 3.0 moles on the product in the control cell line) on the expressed recombinant protein .

### Example V

The cell lines of Examples III and IV are used in overexpression of a sialyltransferase and galactosyl transferase according to the methods of Example I.

The cell line is used in the production of recombinant glycoprotein.

What is claimed is:

1. A process for producing a glycoprotein in a eukaryotic cell capable of expressing the glycoprotein comprising the steps of:
introducing into the eukaryotic cell at least a first and a second gene capable of being expressed by the eukaryotic cell the first gene comprising a nucleic acid sequence encoding a galactosyltransferase and the second gene comprising a nucleic acid sequence encoding a sialyltransferase;
maintaining the eukaryotic cell under conditions suitable for the expression of the galactosyltransferase, the sialyltransferase and the glycoprotein.

2. The process according to claim 1 wherein the glycoprotein is a heterologous glycoprotein.

3. The process according to claim 2 wherein the eukaryotic cell is a Chinese hamster ovary (CHO) cell.

4. The process according to claim 3 wherein the heterologous glycoprotein is a human glycoprotein.

5. The process according to claim 4 wherein the sialyltransferase is an α2,3-sialyltransferase.

6. The process according to claim 5 wherein the galactosyltransferase is a β1, 4-galactosyltransferase.

7. The process according to claim 1 wherein the eukaryotic cell is selected for reduced functional expression of a sialidase.

8. The process according to claim 7 wherein the eukaryotic cell expresses a sialidase antisense RNA.

9. The process according to claim 7 wherein the eukaryotic host cell displays a decreased expression of sialidase RNA.

10. The process according to claim 7 wherein the glycoprotein is a heterologous glycoprotein.

11. The process according to claim 10 wherein the eukaryotic cell is a Chinese hamster ovary (CHO) cell.

12. The process according to claim 11 wherein the heterologous glycoprotein is a human glycoprotein.

13. The process according to claim 12 wherein the sialyltransferase is an α2,3-sialyltransferase.

14. The process according to claim 13 wherein the galactosyltransferase is a β1,4-galactosyltransferase.
